(19) United States
(12) Patent Application Publication
(10) Pub. No.: US 2021/0001159 A1
(43) Pub. Date: Jan. 7, 2021

(54) SMELL ELIMINATOR

(71) Applicant: Mohammed Al Wandy, Las Vegas, NV (US)

(72) Inventor: Mohammed Al Wandy, Las Vegas, NV (US)

(21) Appl. No.: 16/502,982

(22) Filed: Jul. 3, 2019

(51) Int. Cl. A62B 23/06; A61M 11/00; A61M 15/08; A61M 21/00; A41D 13/11; A62B 23/02; A62B 18/02

(52) U.S. Cl. CPC A62B 23/06; A61M 11/008; A61M 15/08; A61M 21/00; A61M 2021/0016; A62B 23/02; A62B 18/025; A61M 2210/0618; A61M 2209/06; A41D 13/11

(57) ABSTRACT

A system for eliminating odor is presented. The system has a nostril plug filter for inserting in to nostril barring odor. The system also has a bottle that has a liquid, a top and an inside. The bottle is squeezed to release a mist of the liquid. The mist from the liquid being sprayed upon the nostril plug filter. The nostril plug filter is inserted into a nostril of a person to eliminate smelling of the odor. The liquid may be scented, such as lavender, peppermint, vanilla, floral, etc. Further, the system may include a container, such as a case or a tube, for storing the nostril plug filter and protecting it from dirt, damage or being lost.

### FIELD OF THE INVENTION

The present invention relates generally to the nose plugs, and more particularly to a smell eliminating nose plug.

### BACKGROUND

The human nose is the most protruding part of the face that bears the nostrils and is the first organ of the respiratory system. The nose is also the principal organ in the olfactory system. The shape of the nose is determined by the nasal bones and the nasal cartilages, including the nasal septum which separates the nostrils and divides the nasal cavity into two. On average the nose of a male is larger than that of a female.

The main function of the nose is respiration, and the nasal mucosa lining the nasal cavity and the paranasal sinuses carries out the necessary conditioning of inhaled air by warming and moistening it. Nasal conchae, shell-like bones in the walls of the cavities play a major part in this process. Filtering of the air by nasal hair in the nostrils prevents large particles from entering the lungs. Sneezing is a reflex to expel unwanted particles from the nose that irritate the mucosal lining. Sneezing can transmit infections, because aerosols are created in which the droplets can harbor pathogens.

Another major function of the nose is olfaction, the informing of odors that the sense of smell carries out. The area of olfactory epithelium, in the upper nasal cavity contains specialized olfactory cells responsible for this function.

When odors are especially strong, melodious or rancid, one can have a gag reaction or adverse reaction to the smell. Therefore, there is a need to have a nose plug that can provide pleasant smells blocking and/or replacing the melodious odors.

### BRIEF DESCRIPTION OF THE DRAWINGS

FIG. 1 is an illustrated view of a nostril plug filter and container.

FIG. 2 is an illustrated detailed view of the nostril plug filter of FIG. 1.

FIG. 3A is an illustrated first view of the system for eliminating a smell utilizing the nostril plug filter of FIG. 1.

FIG. 3B is an illustrated second view of the system for eliminating a smell utilizing the nostril plug filter of FIG. 1.

FIG. 3C is an illustrated third view of the system for eliminating a smell utilizing the nostril plug filter of FIG. 1.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

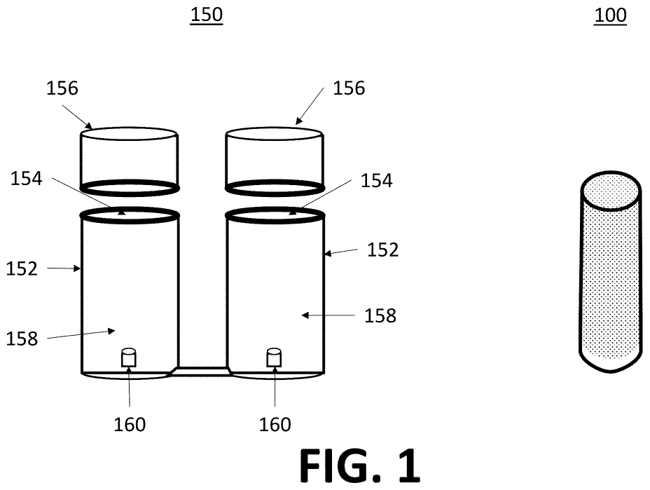

Referring to FIG. 1, an illustrated view of a nostril plug filter 100 and a container 150 for the nostril plug filter 100 is presented. The nostril plug filter 100 is useful for preventing, at least, some amount of odor or smog from entering the nasal passages of a person. The container 150 is useful for storing the nostril plug 100. The nostril plug filter 100 is preferably made of a dense foam, but may be rubber, etc. The nostril plug filter 100 may be a single piece, but it may have one or more compartments for storing a liquid. The liquid is preferably a scented liquid, but it may be water, etc. The nostril plug filter 100 may be a size of large, medium or small to accommodate various users.

The container 150 has an outside 152, an inside 154, a cap 156, a lower portion 158 and an airhole. The container 150 is useful for storing the nostril plug filter 100 and for keeping the nostril plug filter 100 from being contaminated, subject to dirt, or lost. The container 150 is preferably made of a hard-plastic material, but any material such as aluminum, steel, etc. may be used as desired.

The container 150 is configured to contain a nostril plug filter 100. The cap 156 of the container 150 may be opened, thus exposing the inside 154 of the container 150. The nostril plug filter 150 is placed into the inside 154 of the container 150. The cap 156 is closed, thus engaging with the lower portion 158 of the container 150. The air hole 160 provides air into the inside 154 of the container 150 such that the nostril plug filter 100 does not dry out or become moldy. The airhole 160 is configured to be on the lower portion 158 of the container 150.

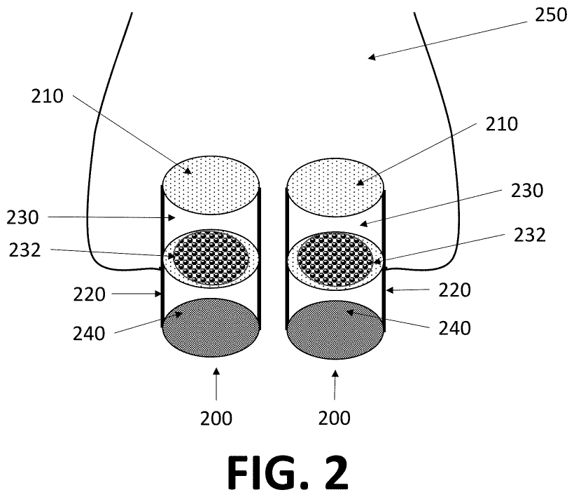

Moving now to FIG. 2, an illustrated a second embodiment of a nostril plug filter 200 of FIG. 1 is presented.

The nostril plug filter 200 has an inside 210, an outside 220, a first compartment 230, a second compartment 240. The outside 220 of the nostril plug filter 200 is made of a soft foam rubber, but may be other material as desired and must be pliable and soft. The nostril plug filter 200 may be a size of large, medium or small to accommodate various users.

The inside 210 of the nostril plug filter 200 has the first compartment 230 and the second compartment 240. The first compartment 230 is useful in being configured to contain one or more fillets 232. The fillets 232 are a solid piece, similar to a wafer, that can emit a scent when being wetted with a liquid.

The second compartment 240 is coupled to the first compartment 230. The second compartment 240 is configured to hold a liquid. The liquid is preferably a scented liquid but may be other types of liquid, including but not limited to, water. The scented liquid may be peppermint, vanilla, floral, lavender, etc. The liquid may induce a smell or scent from the fillets of the first compartment 330 of the nostril plug filter 200.

The outside 220 of the nostril plug filter 200 must be configured to allow the scent to be released outside of the nostril plug filter 200 and also allow the liquid to be spread on the outside 220 of the nostril plug filter 200 and be stored in the second compartment 240 of the inside 210 of the nostril plug filter 200.

The nostril plug filter 200 may be configured to be effective with a mask 250. The mask 250 is useful for those that wish to not enter a nostril plug filter 100 into their nostrils. Instead the mask 250 fits over a nose of the person and is configured to accept the nostril plug filter 200. Therefore, the nostril plug filter 200 does not come in contact with the nostril of the person but instead provides the scent without contact. The mask is preferably made of a clear plastic material, but may be made of other material as desired.

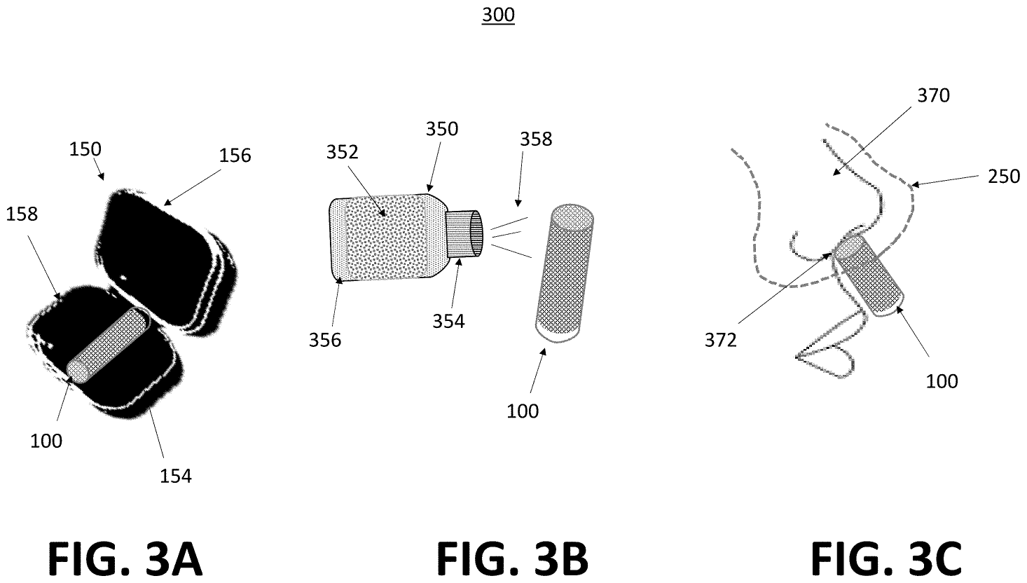

In FIG. 3A, FIG. 3B and FIG. 3C, an exemplary system 300 for providing a smell eliminator is presented. The system 300 is useful for providing a barrier to melodious odors that normally permeate the nasal passages of a person and may cause adverse reactions such as a gag reaction, etc.

In FIG. 3A, the nostril plug filter 100 of FIG. 1 is shown in a container 150. The top 156 of the container 150 is decoupled from the lower portion 158 of the container 150. The nostril plug filter 100 is stored in the inside 154 of the container 150. The nostril plug filter 100 is removed from the container 150. The container 100 is preferably a case, but may also be a tube, etc.

FIG. 3B illustrates the nostril plug filter 100 of FIG. 1 being prepared to eliminate odor in the air from the nasal passage of a person. The liquid bottle 350 has a top 354, an inside 356 and a liquid 352. The liquid bottle 350 contains a liquid 352.

The user desires to spray the liquid 352 from the liquid bottle 350 with a scent onto the nostril plug filter 100. Once the top 354 has been opened or unscrewed, the user squeezes the liquid bottle 350 forcing the liquid 352 contained on the inside 356 of the liquid bottle 350 into a mist 358.

The user places the nostril plug filter 100 into the path of the mist 356 of the liquid 352 contained in the inside 356 of the liquid bottle 250. The user then decides to utilize the now scented nostril plug filter 100.

In FIG. 3C, the user utilizes the now scented nostril plug filter 100. The user has a nose 370. The nose 370 of the person has two nostrils 372. The mask 250 of FIG. 2 is placed over the nose 370 of the user providing a path to the nostrils 372.

The user inserts the nostril plug filter 100 through the opening in the mask 250. The user can then either leave it outside of the nostril 372 or to insert the nostril plug filter 100 further and enter the nostril 372 with the nostril plug filter 100. The user determines how long they desire or need to have the nostril plug filter 100 inserted into their nostril 372 to eliminate an undesired odor.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A system for eliminating odor, comprising:
a nostril plug filter, the nostril plug filter for inserting in to nostril barring odor;
a bottle, the bottle having a liquid, a top and an inside; and
wherein the bottle being squeezed to release a mist of the liquid and wherein the mist from the liquid being sprayed upon the nostril plug filter; and
wherein the nostril plug filter being inserted into a nostril of a person to eliminate smelling of the odor.

2. The system of claim 1, further comprising:
a mask, the mask for coupling to a face of the user; and
Wherein the nostril plug filter being inserted into an opening in the mask.

3. The system of claim 2, wherein the nostril plug filter inserted into the mask being further inserted into the nostril.

4. The system of claim 2, wherein the mask is a clear plastic material.

5. The system device of claim 1, further comprising:
a container, the container being for storing the nostril plug filter.

6. The system of claim 5, wherein the container being a case.

7. The system of claim 5, wherein the container being a tube.

8. The system of claim 1, wherein the liquid being scented.

9. The system of claim 1, wherein the scent being peppermint.

10. The system of claim 1, wherein the nostril plug filter being a size of one of:
large, medium and small.
